(19) United States
(12) Patent Application Publication
(10) Pub. No.: US 2003/0088224 A1
(43) Pub. Date: May 8, 2003

(54) Absorbent article having message

(76) Inventors: Glory F. Ceman, Appleton, WI (US); Paulette M. Rosch, Sherwood, WI (US); Jan E. Carr, Appleton, WI (US); Donald G. Fox, Lady Lake, FL (US); Lisa R. White, Ogden, UT (US); Tara E. Andrews, Fort Jennings, OH (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/003,077

(22) Filed: Nov. 2, 2001

(51) Int. Cl. A61F 13/20; A61L 15/00; A61B 17/06; A61B 19/02

(52) U.S. Cl. 604/385.01; 604/385.05; 206/438

(57) ABSTRACT

An absorbent article including an absorbent body and an adhesive coating applied to a first side of the body permitting a user to attach the body to a preselected surface. The article also includes a covering removably attached to the adhesive coating to prevent attachment of the body before the user removes the covering from the adhesive coating. The covering has a release side for contacting the adhesive coating and a message side opposite the release side. The message side includes indicia directed to communicating a message to the user related to motivating, entertaining, educating, and/or inspiring the user.

### BACKGROUND OF THE INVENTION

The present invention relates generally to absorbent articles and more particularly to a method of establishing and nurturing a business relationship between an absorbent article seller and a consumer of the articles.

Absorbent articles such as panty liners and feminine napkins frequently include areas coated with adhesive materials for attaching the article to a crotch area of clothing to hold the article in place adjacent a woman's pudendum during use. Frequently, a label or covering is removably attached to the adhesive coating to cover the coating and to prevent attachment of the article before the user removes the covering from the adhesive coating. In the past, messages related to instructions for using the article and/or designations of a source of the article (e.g., a trademark) were printed on the removable coverings. Because most women are aware how to use absorbent articles, the instructions are neither needed nor read each time the articles are used.

Although the instructions are not used usually, there is information which could be beneficial to women if it were included on the coverings. Some women may be unaware of certain health related information. Thus, there is a need to educate women regarding this information. Further, some women experience emotional lows during the times when they are using the articles and may be in need of motivation and/or inspiration. In addition, some women may desire entertainment while using the articles. Including messages intended to motivate, entertain, educate and/or inspire could be beneficial to women.

Some manufacturers and/or sellers of absorbent articles wish to establish and/or nurture their business relationship with their consumers. One way to establish and nurture these relationships is to fulfill the previously identified needs and desires of consumers.

### SUMMARY OF THE INVENTION

Briefly, a method of this invention is for establishing and nurturing a business relationship between a product seller and a consumer. The method comprises attaching a removable label to a product. The label is removed from the product prior to using the product to enable full functionality of the product. Further, the method comprises positioning indicia on the removable label directed to communicating a message to the user related to at least one of motivating the user, entertaining the user, educating the user and inspiring the user. The product is placed inside packaging having instructions related to use of the product.

In another aspect, the method is for establishing and nurturing a business relationship between a seller of absorbent articles and a consumer thereof. A removable label is attached to an absorbent article to cover an adhesive coating applied to the article for attaching the article to a preselected surface. Further, indicia is positioned on the removable label.

In still another aspect, an absorbent article of the present invention comprises an absorbent body having a peripheral edge, a first side, and a second side opposite the first side. The article also comprises an adhesive coating applied to the first side of the body permitting a user to attach the body to a preselected surface and a covering removably attached to the adhesive coating to prevent attachment of the body before the user removes the covering from the adhesive coating. The covering has a size and shape selected for covering at least a portion of the adhesive coating when attached to the adhesive coating, a release side for contacting the adhesive coating when the covering is attached to the coating, and a message side opposite the release side. The message side of the covering includes indicia directed to communicating a message to the user related to motivating, entertaining, educating and/or inspiring the user.

In yet another aspect, the present invention includes a package of absorbent articles comprising a plurality of absorbent articles. At least a portion of the articles has a removable label including indicia thereon. Further, the package includes a container sized and shaped for holding the absorbent articles. The container has indicia thereon for instructing a user of the article regarding use the article.

In a final aspect, the present invention includes a laminated structure comprising a body, an adhesive coating, and a covering removably attached to the adhesive coating. The covering has a release side and a message side. The message side includes indicia directed to communicating a message to the user related to motivating, entertaining, educating and/or inspiring the user.

Other features of the present invention will be in part apparent and in part pointed out hereinafter.

### BRIEF DESCRIPTION OF THE DRAWINGS

FIG. 1 is a top plan of a feminine napkin;

FIG. 2 is a bottom plan of the napkin;

FIG. 3 is a front elevation of an envelope having a folded napkin therein; and

FIG. 4 is a rear elevation of packaging containing envelopes holding folded napkins therein.

Corresponding reference characters indicate corresponding parts throughout the several views of the drawings.

### DETAILED DESCRIPTION OF THE PREFERRED EMBODIMENT

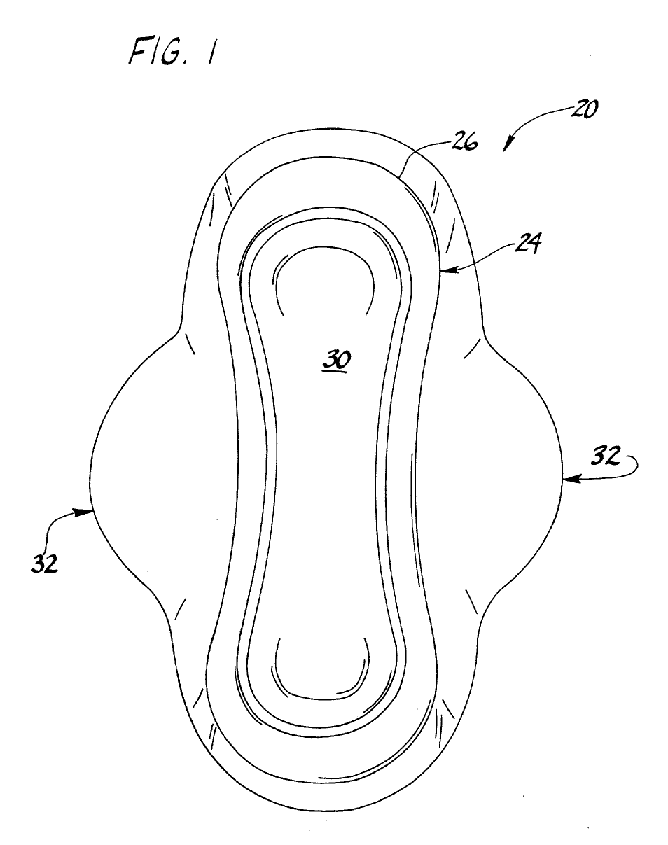

Referring now to the drawings and in particular to FIG. 1, a conventional absorbent article is designated in its entirety by the reference numeral 20. Although the absorbent article 20 shown in FIG. 1 is a feminine napkin, those skilled in the art will appreciate that the present invention may be applied to other absorbent articles 20 such as panty liners, other feminine care products, adult care products, child care products and infant care products.

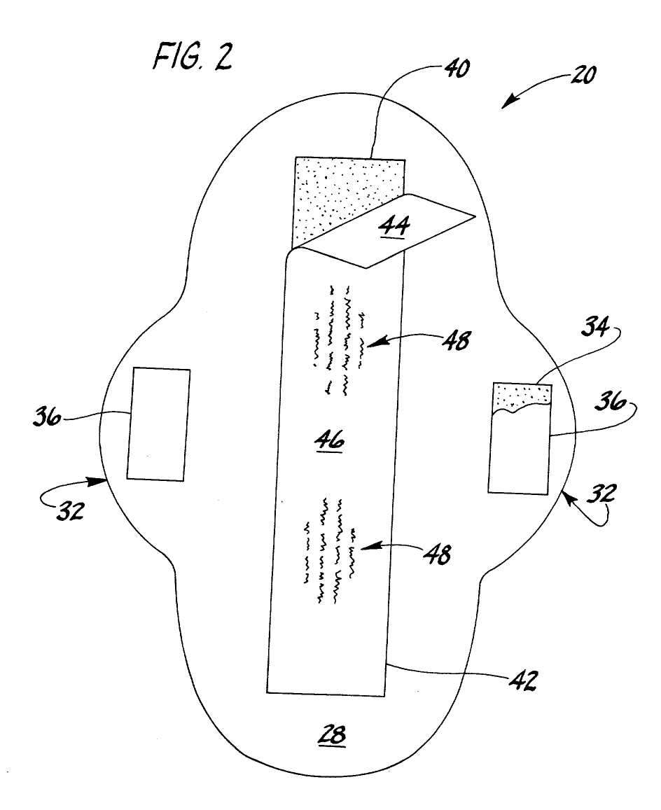

The feminine napkin 20 shown in FIG. 1 is a laminated structure comprising an absorbent body, generally designated by 24, having a peripheral edge 26, a first side 28 (FIG. 2), and a second side 30 opposite the first side. Tabs or flaps, generally designated by 32, extend laterally from each lateral edge of the body 24 for wrapping around the crotch of the user's underwear (not shown) to protect it from staining. As illustrated in FIG. 2, each tab 32 may include an adhesive strip or other fastener 34 for attaching the tab in a conventional manner to an outside surface of a crotch of the user's underwear to hold the flaps 32 of the napkin 20 in place around the underwear during use. The adhesive strips 34 may be covered with a removable peel strip covering 36 to prevent the strips from sticking to other surfaces until ready for use.

As further illustrated in FIG. 2, an adhesive coating 40 (e.g., a two-sided adhesive film or tape) is also applied to the first side 28 of the body 24 permitting a user to attach the body to a preselected surface such as the inside surface of the crotch of the user's underwear to hold the napkin 20 in place on the underwear during use. Although the coating 40 may have other sizes and shapes without departing from the scope of the present invention, in one embodiment the coating is rectangular and has a width of about three centimeters and a length of about 17 centimeters. A covering or label 42 is removably attached to the adhesive coating 40 to prevent attachment of the body 24 before the user removes the covering from the adhesive coating. Although the covering 42 may have other sizes and shapes without departing from the scope of the present invention, in one embodiment the covering has a size and shape selected for entirely covering the adhesive coating 40 when attached to the adhesive coating. More particularly, in one embodiment the covering 42 is rectangular and has a width of about four centimeters and a length of about 17 centimeters. Because the covering 42 is wider than the adhesive coating 40, it presents a loose margin which may be grasped easily by the user when removing the covering from the coating.

As further illustrated in FIG. 2, the covering 42 has a release side 44 for contacting the adhesive coating 40 when the covering is attached to the coating and a message side 46 opposite the release side. As will be explained in greater detail below, the message side 46 of the covering 42 includes indicia, generally designated by 48, thereon. Although the indicia 48 may be applied to the covering 42 in other ways (e.g., embossing) without departing from the scope of the present invention, in one embodiment the indicia are printed on the message side 46 of the covering using conventional printing techniques. All other aspects of the napkin 20 are conventional and well known by those skilled in the art. Thus, the materials used in making the napkin 20 describe above, the detailed construction of the napkin, and the method of manufacturing the napkin are well known and will not described in further detail. One such conventional napkin 20 is described in U.S. Pat. No. 5,429,630, issued Jul. 4, 1995.

The indicia 48 on the message side 46 of the covering 42 is directed to communicating a message to the user. The message is related to at least one of motivating the user, entertaining the user, educating the user, and inspiring the user. Further, the message is intended to establish and/or nurture a business relationship between a seller of the absorbent articles and a consumer. For example, the indicia 48 may communicate an inspirational or motivational message such as “Life is a verb, not a noun.” or “Live each day to its fullest.” Examples of an entertaining message include humorous messages such as “You flow girl.” or fortune-cookie-type messages such as “You will mend a rift with an old friend.” or trivia-type messages such as “Did you know that black is the most popular underwear color among women?” An example of an educational message includes health related messages such as “Remember to drink eight glasses of water each day to stay healthy.” or “Visit your gynecologist annually for good health.” Although the message side 48 may also include messages related to using the absorbent article or messages related to designating a source of the absorbent article, in one embodiment the message side 46 of the covering is substantially free of messages related to using the napkin 20 and messages related to designating a source of the napkin (e.g., the manufacturer's trademark).

As will be appreciated by those skilled in the art, it is desirable to remove the covering 42 from the napkin 20 to apply the napkin to underwear and thereby to enable full functionality of the napkin. Thus, the user comes in physical contact with the covering 42 desirably drawing the user's attention to the message. This is particularly beneficial when relaying messages such as those related to health issues. Therefore, the placement of messages of the types described above on the message side 48 of the covering 42 is of particular significance to the present invention.

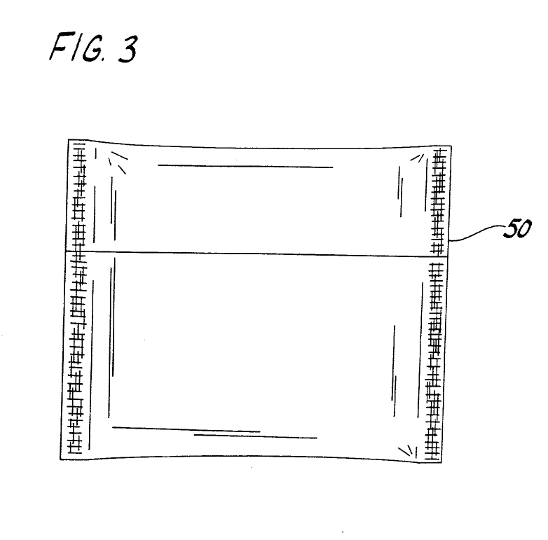

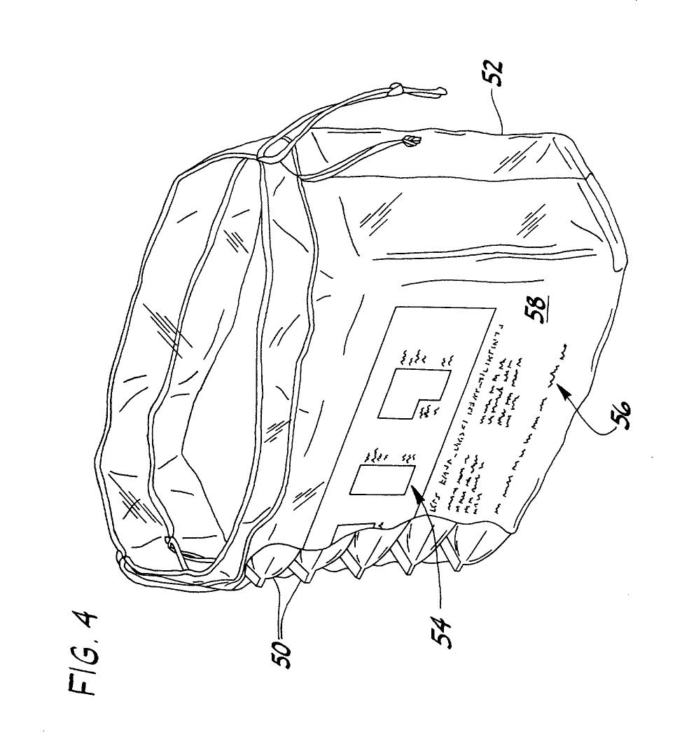

Although the napkin 20 may be packaged in other ways without departing from the scope of the present invention, in one embodiment the napkin is folded and placed in an envelope or wrapper or other individual packaging 50 as shown in FIG. 3. As the envelope 50 is conventional, it will not be described in detail. After being placed in the envelope 50, in one embodiment the napkin 20 is grouped with several other napkins and the grouped napkins are placed in a retail container or package 52 such as soft or hard packaging. Soft packaging includes flexible envelopes and packages made of sheet plastic and paper. Hard packaging includes generally less flexible packages made of plastic (e.g., tubs and buckets), metal and cardboard. In one embodiment, the package 52 is a conventional reclosable draw string package as shown in FIG. 4. As further illustrated in FIG. 4, in one embodiment the package 52 includes a message, generally designated by 54, related to using the absorbent article (i.e., instructions of use) and a message, generally designated by 56, related to a designation of a source of the product (e.g., a trademark or trade name). Although the message may be positioned on or in the package 52 in any conventional way without departing from the scope of the present invention, in one embodiment the message 54 is printed on an exterior surface 58 of the package.

Other packaging is envisioned. For example, in some instances it may be desirable to package the napkin 20 in only an envelope 50 as described above and to omit the outer packaging. In that instance, the envelope 50 would constitute the entire product packaging. In other instances, it may be desirable to package several absorbent articles 20 in a conveniently sized package (not shown) for daily use and then package these packages in larger retail packages. Further, it may be desirable to package absorbent articles 20 of different types and/or sizes or those having different absorbencies together in a single package. It is also envisioned it may be desirable to package absorbent articles 20 having different messages, different types of messages, and/or no message at all in a single package.

As will be appreciated by those skilled in the art, the absorbent article 20 and packaging described above may be used to establish and nurture a business relationship between a product seller and a consumer. To establish and nurture this relationship, the product manufacturer, distributor or seller attaches a removable label to the product. In one embodiment the label is removed from the product prior to using the product to enable full functionality of the product as described above. For example, the label may cover an adhesive coating applied to the article for attaching the article to a preselected surface. Indicia is positioned on the label. The indicia is directed to communicating a message to the user related to motivating the user, entertaining the user, educating the user, and/or inspiring the user. The product is placed inside packaging. In one embodiment, the packaging has instructions related to use of the product.

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A method of establishing and nurturing a business relationship between a product seller and a consumer comprising:
attaching a removable label to a product, wherein said label is removed from the product prior to using the product to enable full functionality of the product;
positioning indicia on the removable label directed to communicating a message to the user related to at least one of motivating the user, entertaining the user, educating the user, and inspiring the user; and
placing said product inside packaging having instructions related to use of the product.

2. A method as set forth in claim 1 wherein said label is substantially free of messages related to using the product.

3. A method as set forth in claim 1 further comprising the step of printing said instructions on an exterior surface of the packaging.

4. A method of establishing and nurturing a business relationship between a seller of absorbent articles and a consumer thereof comprising:
attaching a removable label to an absorbent article to cover an adhesive coating applied to the article for attaching the article to a preselected surface;
positioning indicia on the removable label directed to communicating a message to the user related to at least one of motivating the user, entertaining the user, educating the user, and inspiring the user; and
placing said article inside packaging.

5. A method as set forth in claim 4 wherein said label is substantially free of messages related to using the article.

6. A method as set forth in claim 4 wherein said packaging has instructions related to use of the article.

7. A method as set forth in claim 6 further comprising printing said instructions on an exterior surface of the packaging.

8. An absorbent article comprising:
an absorbent body having a peripheral edge, a first side, and a second side opposite said first side;
an adhesive coating applied to said first side of the body permitting a user to attach said body to a preselected surface; and
a covering removably attached to the adhesive coating to prevent attachment of the body before the user removes the covering from the adhesive coating, said covering having a size and shape selected for covering at least a portion of the adhesive coating when attached to the adhesive coating, a release side for contacting the adhesive coating when the covering is attached to the coating, and a message side opposite said release side;
wherein said message side of the covering includes indicia directed to communicating a message to the user related to at least one of motivating the user, entertaining the user, educating the user, and inspiring the user.

9. An absorbent article as set forth in claim 8 wherein said message side of the covering is substantially free of messages related to using the absorbent article.

10. An absorbent article as set forth in claim 9 wherein:
said absorbent article is a first absorbent article;
said first absorbent article is grouped with a second absorbent article; and
said first and second absorbent articles are received in a container.

11. An absorbent article as set forth in claim 10 wherein said second absorbent article is substantially identical to said first absorbent article.

12. An absorbent article as set forth in claim 10 wherein the container includes a message related to using the absorbent article.

13. An absorbent article as set forth in claim 12 wherein the message related to using the absorbent article is printed on an exterior of the container.

14. An absorbent article as set forth in claim 8 wherein said message side of the covering is substantially free of messages related to designating a source of the absorbent article.

15. An absorbent article as set forth in claim 14 wherein:
said absorbent article is a first absorbent article;
said first absorbent article is grouped with a second absorbent article; and
said first and second absorbent articles are received in a container.

16. An absorbent article as set forth in claim 15 wherein said second absorbent article is substantially identical to said first absorbent article.

17. An absorbent article as set forth in claim 15 wherein the container includes a message related to using the absorbent article.

18. An absorbent article as set forth in claim 17 wherein the message related to using the absorbent article is printed on an exterior of the container.

19. An absorbent article as set forth in claim 8 wherein the absorbent body is a feminine napkin.

20. A package of absorbent articles comprising:
a plurality of absorbent articles, at least a portion of said plurality of absorbent articles having a removable label including indicia thereon related to at least one of motivating, entertaining, educating and inspiring a user of the article; and
a container sized and shaped for holding said plurality of absorbent articles, said container having indicia thereon for instructing a user of the article regarding use the article.

21. A package as set forth in claim 20 wherein said label is removed from each of said absorbent articles having a label to enable full functionality of the respective article.

22. A package as set forth in claim 21 wherein said label covers an adhesive coating for attaching the article to clothing and thereby ensure proper placement of the article during use.

23. A package as set forth in claim 20 wherein the removable labeling is substantially free of messages related to using the absorbent article.

24. A package as set forth in claim 20 wherein the removable labeling is substantially free of messages related to designating a source of the absorbent article.

25. A laminated structure comprising:
a body having a peripheral edge, a first side, and a second side opposite said first side;
an adhesive coating applied to said first side of the body permitting a user to attach said body to a preselected surface; and
a covering removably attached to the adhesive coating to prevent attachment of the body before the user removes the covering from the adhesive coating, said covering having a size and shape selected for covering at least a portion of the adhesive coating when attached to the adhesive coating, a release side for contacting the adhesive coating when the covering is attached to the coating, and a message side opposite said release side;
wherein said message side of the covering includes indicia directed to communicating a message to the user related to at least one of motivating the user, entertaining the user, educating the user, and inspiring the user.
